(19) United States
(12) Patent Application Publication
(10) Pub. No.: US 2020/0276429 A1
(43) Pub. Date: Sep. 3, 2020

(54) TATTOO NEEDLE TIP WITH A CAPILLARY INK RESERVOIR AND COMBINED DEVICE THEREOF

(71) Applicant: LONG XIAO, Scarborough (CA)

(72) Inventor: LONG XIAO, Scarborough (CA)

(21) Appl. No.: 16/878,348

(22) Filed: May 19, 2020

Related U.S. Application Data
(63) Continuation-in-part of application No. 15/123,464, filed on Sep. 2, 2016.
Application No. 15/123,464 is a continuation-in-part of application No. 15/123,464, filed on Sep. 2, 2016.
Application No. 15/123,464 is a continuation-in-part of application No. 15/874,597, filed on Jan. 18, 2018.

(30) Foreign Application Priority Data
Apr. 8, 2014 (CN) 201420167938.8

(51) Int. Cl. A61M 37/00

(52) U.S. Cl. CPC A61M 37/0084; A61M 2210/04

(57) ABSTRACT

A tattoo needle tip with a capillary ink reservoir and combined device thereof are provided. The tattoo needle tip includes a needle tip body. A needle passage is defined inside the needle tip body for placing a tattoo needle. A needle opening is defined at one end of the needle tip body for the sharp end of the tattoo needle to extend out. The needle opening is communicated with the needle passage. A capillary ink reservoir adapted to receive and retain ink therein by capillary action is disposed on the needle tip body and directly connected to the needle opening whereby the ink reservoir can directly feed ink to the sharp end of the tattoo needle. A needle assembly for a liquid applicator is also provided, in which the needle is biased towards ink storage grooves of the ink reservoir.

### CROSS-REFERENCE TO RELATED APPLICATIONS

This application is a continuation-in-part application of U.S. patent application Ser. No. 15/123,464, filed Sep. 2, 2016, entitled “Tattoo Needle Tip with a Capillary Ink Reservoir and Combined Device Thereof”, which is a national stage application of PCT International Application No. PCT/CN2015/075980, which is filed Apr. 7, 2015 and claims the benefit of, and priority from, Chinese Invention Patent Application No. 201420167938.8, filed Apr. 8, 2014, the entire contents of each of which are incorporated herein by reference.

This application is also a continuation-in-part and, claims the benefit of, and priority from, U.S. patent application Ser. No. 15/874,597, filed Jan. 18, 2018, the entire contents of which are incorporated herein by reference.

### FIELD

The present disclosure relates to an application device for tattooing or for making permanent make-up, particularly relates to a tattoo needle tip with a capillary ink reservoir and its combined devices with a handle and with a tattoo needle, and devices for applying a liquid to skin, particularly to disposable needle assemblies utilizing capillary action for feeding ink or another liquid in such devices.

### BACKGROUND

A conventional tattoo device generally includes a tattoo machine, a handle, a needle tip and a tattoo needle. The user assembles these components together before use. While assembling, the handle is connected to the tattoo machine, the needle tip is placed on the handle, the tattoo needle passes through the central hole of the handle with the sharp end of the needle thereof reaching the opening of the tattoo needle tip.

In prior art, the basic shape of the needle tip is essentially a cylindrical tube. One end of the needle tip has a frustum section with a needle passage being defined therethrough. The free end of the frustum section is provided with a needle opening for the tattoo needle extending out.

The interior of the frustum section of the needle tip forms an inner cavity in a shape of a frustum. The inner cavity is communicated with the needle opening. The inner walls of the inner cavity and the needle opening are mostly designed to be streamlined and smooth, so as to prevent the sharp end of the needle from resisting when the tattoo needle is inserted thereby preventing the sharp end of the needle from bending. Meanwhile the inner cavity also serves as an ink reservoir.

During operation, the tattoo needle is doing a straight-line reciprocating motion in high frequency. The sharp end of the needle wetted by tattoo ink rushes out from the needle opening and stabs into the skin, then the sharp end of the needle returns into the needle opening for dipping some ink and then rushes out again. The section of the needle opening for feeding ink to the sharp end of the needle is the feed section. When adding ink to the needle tip, the tattooist inserts the needle tip into a small ink cup. There is a gap between the inner wall of the needle opening and the tattoo needle. Generally the ink will go into the needle opening and moisturize the needle opening and the inner wall of the needle passage. Some tattooists will briefly start the tattoo machine at the same time as dipping ink, then the tattoo needle does an up and down reciprocating motion in high speed, which will raise the ink to fill the inner cavity of the frustum section. In tattooing, the smooth inner wall of the inner cavity in the needle tip cannot effectively retain the ink. Therefore, the ink will flow to the needle opening due to the gravity and the motion of the tattoo needle.

The common issue met by tattooists when using these needle tips is that, if too much ink is added to the needle tip, it is easy to cause leakage of ink and pollute the tattoo during tattooing. In order to avoid the leakage of ink, tattooists have to add little ink each time, which increases the frequency of adding ink and results in ink failure or insufficient ink provision. However, it is inconvenient for tattooists to add ink frequently in tattooing. The frequent occurrence of ink failure or insufficient ink provision will adversely affect tattooists in drawing fluent and long tattoo lines.

U.S. Patent No. 20130226211 A1 and Chinese patent No. 202538157U disclose a needle tip with a capillary ink reservoir. The ink reservoir has functions of ink storage and ink maintenance, whereby once dipping ink, the tattoo needle tip can keep tattooing for a longer time. Thus, tattooists can avoid dipping ink frequently when tattooing. The capillary ink reservoir is connected to the needle opening via an ink guide channel. There is a certain distance from the capillary ink reservoir to the needle opening. To quickly fill the capillary ink reservoir with ink, tattooists need to immerse the ink reservoir in the small ink cup with a deeper insertion when dipping ink. Since the deeper insertion leads to too much ink on the outside surface of the needle tip, tattooists have to constantly wipe off the ink on the outside surface of the needle tip. Due to the ink guide channel spaced between the ink reservoir and the needle opening, it is not easy to supply ink with poor mobility and high viscosity to the needle opening timely and sufficiently. There is a phenomenon that some amount of ink left in the capillary ink reservoir cannot flow to the needle opening, which results in the low efficiency of the capillary ink reservoir. The ink guide channel cannot adapt well to ink of different viscosities at the same time. For ink of low viscosity, a gap size of the ink guide channel shall be smaller, which will cause ink of high viscosity from failing to flow through the channel fluently. Thus, the gap size of the ink guide channel should be bigger for ink of high viscosity, which will cause flooding or leakage of ink of low viscosity.

For safety reasons, the needle is typically sterilized before use and replaced after each use. It is thus convenient to use replaceable and disposable needle modules or needle tips, which can be pre-sterilized and easily detached or connected to the needle handle so that the needle can be easily replaced, and safely disposed. Disposable needle modules, particularly single-use disposable needle modules or similar disposable needle assemblies are thus often used with tattooing devices and other similar devices. Devices or applicators for applying permanent mark-up may have a similar construction, with a base, a replaceable handle, and a replaceable/disposable needle module. There are a number of drawbacks in existing disposable needle assemblies.

### SUMMARY

An aspect of the present disclosure relates to solving one or more problems present in the existing ink feeding techniques used for feeding ink in a tattooing device.

It is desirable to provide a tattoo needle tip with a capillary ink reservoir, which has functions of ink storage and ink maintenance, does not easily cause leakage of ink, and insures rapid filling of the ink reservoir, also insures effective feeding of the ink to the sharp end of the tattoo needle during tattooing.

It is also desirable to provide a combined device comprising a tattoo needle tip with a capillary ink reservoir and a handle, which forms a combined component of a tattoo device, has functions of ink storage and ink maintenance, does not easily cause leakage of ink, and insures rapid filling of the reservoir, also insures effective feeding of the ink to the sharp end of the tattoo needle during tattooing.

It is further desirable to provide a combined device comprising a tattoo needle tip with a capillary ink reservoir and a tattoo needle, which forms a combined component of a tattoo device, has functions of ink storage and ink maintenance, does not easily cause leakage of ink, and insures rapid filling of the reservoir, also insures effective feeding of the ink to the sharp end of the tattoo needle during tattooing.

In an embodiment of the present disclosure, a tattoo needle tip with a capillary ink reservoir of the present disclosure includes a needle tip body. A needle passage is defined inside the needle tip body for placing a tattoo needle. A needle opening is defined at one end of the needle tip body for the sharp end of the tattoo needle to extend out. The needle opening is communicated with the needle passage. A capillary ink reservoir adapted to receive and retain ink therein by capillary action is disposed on the needle tip body and directly connected to the needle opening whereby the ink reservoir can directly feed ink to the sharp end of the tattoo needle. A plurality of capillary ink containing bodies may be provided at the ink reservoir and be directly connected to the needle opening. Each ink containing body may be a capillary aperture defined in the ink reservoir. All the capillary apertures defined in the ink reservoir may be connected to the needle opening directly. The ink reservoir may be made of porous material or fibrous material, and the ink containing bodies may include pores in the ink reservoir or gaps between fibers. The ink reservoir may be integrally or detachably formed at the needle tip body.

In another embodiment, a combined device includes a handle and a tattoo needle tip with a capillary ink reservoir installed to the handle. The tattoo needle tip includes a needle tip body. A needle passage is defined inside the needle tip body for placing a tattoo needle. A needle opening is defined at one end of the needle tip body for the sharp end of the tattoo needle to extend out. The needle opening is communicated with the needle passage. A capillary ink reservoir adapted to receive and retain ink therein by capillary action is disposed on the needle tip body and directly connected to the needle opening whereby the ink reservoir can directly feed ink to the sharp end of the needle.

A combined device in accordance with a further embodiment includes a tattoo needle tip with a capillary ink reservoir and a tattoo needle installed therein. The tattoo needle tip includes a needle tip body. A needle passage is defined inside the needle tip body for placing the tattoo needle. A needle opening is defined at one end of the needle tip body for the sharp end of the tattoo needle to extend out. The needle opening is communicated with the needle passage. A capillary ink reservoir adapted to receive and retain ink therein by capillary action is disposed on the needle tip body and directly connected to the needle opening whereby the ink reservoir can directly feed ink to the sharp end of the needle. A plurality of capillary ink containing bodies may be provided at the ink reservoir and be directly connected to the needle opening. Each ink containing body may be a capillary aperture defined in the ink reservoir. All the capillary apertures defined in the ink reservoir may be connected to the needle opening directly. The ink reservoir may be made of porous material or fibrous material, and the ink containing bodies may include pores in the ink reservoir or gaps between fibers. The ink reservoir may be integrally or detachably formed at the needle tip body.

By locating the ink reservoir at the needle tip, the capillary principle is utilized to store ink in the ink reservoir, which can store and retain a great amount of ink. Therefore, the leakage of ink does not easily happen. The ink containing body of the ink reservoir is connected to the needle opening directly, and the sharp end of the needle continuously dips the ink in the needle opening via the reciprocating motion of the tattoo needle, realizing that dipping and adding ink once, the tattoo needle tip keeps tattooing for a longer time, so as to avoid dipping and adding ink frequently when tattooists are tattooing. Moreover, it is quick and easy to dip and add ink to the ink reservoir.

In an aspect, there is provided a tattoo needle tip comprising a needle tip body having a lower end and an upper end; a needle passage defined inside the needle tip body for placing a tattoo needle; a needle opening defined at the lower end of the needle tip body for a sharp end of the tattoo needle to extend out, the needle opening being communicated with the needle passage; a capillary ink reservoir receiving and retaining ink therein by capillary action being disposed on the needle tip body and directly connected to the needle opening, the ink reservoir directly feeding ink to the sharp end of the tattoo needle, wherein a plurality of capillary ink containing bodies are provided at the ink reservoir and are directly connected to the needle opening.

Further, it has been recognized that in some cases, even when there is still some or a substantial amount of ink in the ink reservoir, little or no ink is supplied to the needle nozzle opening or the sharp end of the needle. More efficient utilization of the ink in the ink reservoir is desirable. Further, residual ink left in the ink reservoir also makes it difficult to clean the ink reservoir and switch ink color during use. It has been surprisingly discovered that ink feeding by capillary action may be improved by increasing direct contact between the needle and the ink in the ink reservoir or the capillary ink feeding channel. Test results have shown that a larger contact area between the needle and ink in the ink reservoir provides better feeding performance. Lack or limited contact between the needle and the ink in the ink reservoir or feeding channel causes inconsistent ink flow and excessive residual ink.

Thus, in a further aspect of the present disclosure, a needle assembly comprises a tattoo needle tip described herein and a tattoo needle mounted in the needle passage of the tattoo needle tip. In this needle assembly, the needle passage comprises a tubular longitudinal channel extending between the lower end and the upper end of the needle tip body, and a guide surface adjacent to the needle opening at the lower end of the needle tip body; the capillary ink containing bodies comprise a plurality of grooves extending generally transversely in the guide surface; a biasing member configured to bias the tattoo needle towards the guide surface across the grooves such that, during longitudinal reciprocal movement of the sharp end of the tattoo needle through the needle opening, contact or a capillary gap between the sharp end of the tattoo needle and the guide surface is maintained to continuously draw ink out of the grooves by capillary action. The needle assembly may be provided in the form of a needle module or a needle cartridge.

There is also disclosed a needle assembly for a liquid applicator such as an ink applicator. The needle assembly includes a housing comprising a tubular longitudinal channel and a guide surface. The channel has an upper open end and a lower open end. A plurality of liquid storage grooves for storing liquid therein by capillary action extend generally transversely in the guide surface above the lower open end. A needle bundle is mounted in the housing. The needle bundle includes one or more needles longitudinally reciprocally movable in the channel and is biased against the guide surface across the grooves such that, during longitudinal reciprocal movement of the needle bundle, the needle bundle maintains contact with the guide surface and the liquid stored in the grooves for drawing the liquid out of the grooves by capillary action. The liquid may include ink.

Conveniently, the contact between the needle bundle and the liquid in the liquid storage grooves can be consistently maintained by the biasing force to provide stable and efficient liquid feeding.

The needle assembly may comprise a biasing member configured to bias the needle bundle to move longitudinally and against the guide surface. The biasing member may comprise a resilient band. The biasing member may comprise a silicone, latex, or rubber material. The guide surface may be shaped to conform to an external profile of the needle bundle to increase contact area between the needle bundle and the liquid stored in the grooves. The needle assembly may comprise a plurality of needles welded or soldered to one another. The needle bundle may comprise 1 to 27 needles. The housing may comprise a generally cylindrical or conical body having a circumference, and one or more of the grooves may extend up to 120° along the circumference of the body. The longitudinal channel may have a generally circular profile, or a generally polygonal profile. The grooves may have a width of about 0.3 mm to about 0.6 mm. The plurality of grooves may comprise 3 to 12 grooves. The housing may comprise a generally conical lower portion and a generally cylindrical upper portion. The grooves may be separated from one another. The grooves may be inter-connected. The housing may comprise an observation window opposite the guide surface, and the needle bundle may comprise a shaft portion and a needle tip portion (needle head), where the shaft portion has a first longitudinal axis and the needle tip portion (needle head) has a second longitudinal axis, which is offset from the first axis towards the observation window.

In another aspect, there is provided a needle assembly for a liquid applicator, comprising a housing comprising a tubular longitudinal channel and an inner surface, the channel comprising an upper open end and a lower open end, a plurality of liquid storage grooves for storing liquid therein by capillary action extending generally transversely in the inner surface above the lower open end; a needle bundle mounted in the housing, the needle bundle comprising one or more needles longitudinally reciprocally movable in the longitudinal channel and being biased towards the inner surface across the grooves to form a capillary gap between the needle bundle and the inner surface for drawing liquid out of the liquid storage grooves during longitudinal reciprocal movement of the needle bundle across the grooves.

In a further aspect, there is provided a liquid applicator such as an ink applicator, which comprises a needle assembly disclosed herein. The liquid applicator may comprise a needle actuator for actuating downward movement of the needle bundle, and a handle coupling the needle actuator to the needle assembly. The liquid applicator may comprise a tattooing device.

Other aspects, features, and embodiments of the present disclosure will become apparent to those of ordinary skill in the art upon review of the following description of specific embodiments in conjunction with the accompanying figures.

### BRIEF DESCRIPTION OF THE DRAWINGS

In the figures, which illustrate, by way of example only, embodiments of the present disclosure:

FIG. 1 is a perspective view of a tattoo needle tip with a capillary ink reservoir according to one embodiment of the present disclosure;

FIG. 2 is a partial section view of FIG. 1;

FIG. 3 is a perspective view of a combined device including a tattoo needle tip with a capillary ink reservoir and a handle according to one embodiment of the present disclosure;

FIG. 4 is a perspective view of a combined device including a tattoo needle tip with a capillary ink reservoir and a handle according to another embodiment of the present disclosure;

FIG. 5 is a partial section view of FIG. 4;

FIG. 6 is a perspective view of a combined device including a tattoo needle tip with a capillary ink reservoir and a handle according to a further embodiment of the present disclosure;

FIG. 7 is a perspective view of a combined device including a tattoo needle tip with a capillary ink reservoir and a tattoo needle according to one embodiment of the present disclosure;

FIG. 8 is a partial section view of FIG. 7;

FIG. 9 is a perspective view of a combined device including a tattoo needle tip with a capillary ink reservoir, a handle, a tattoo needle and a tattoo machine according to the present disclosure;

FIG. 10A is a right side view of the partially-cut device of FIG. 7, with the partial section view cut along the axial central line and with the needle being centrally positioned;

FIG. 10B is a front elevation view of the device of FIG. 10A;

FIG. 10C is the same view as in FIG. 10A, except the needle being biased downward towards the inner surface of the needle passage;

FIG. 10D is a front elevation view of the needle tip of FIG. 10C;

FIG. 11 is a front elevation view of a disposable needle module, illustrative of another embodiment of the present disclosure;

FIG. 12 is an exploded perspective view of the needle module of FIG. 11;

FIG. 13A is a side cross-sectional view of the needle module of FIG. 11;

FIG. 13B is a side elevation view of certain parts of the needle module shown in FIG. 13A;

FIG. 13C is a force diagram showing the force applied to the needle bundle shown in FIG. 13B;

FIG. 14A is a partially-cut perspective view of the needle module of FIG. 11;

FIG. 14B is an axial cross-sectional view of the needle module of FIG. 14A, taken along line 14B-14B in FIG. 14A;

FIG. 14C is an enlarged view of the portion 14C of the needle module of FIG. 14A;

FIG. 14D is an enlarged view of the portion 14D of the needle module of FIG. 14B;

FIGS. 15A, 15B, 15C, 15D and 15E are axial cross-sectional views of various variants of the needle module, illustrative different embodiments of the present disclosure;

FIG. 16 is a side elevation view of a tattooing device, with partial cut-away cross-sectional view of the needle handle and needle module in the tattooing device, illustrative embodiments of the present disclosure;

FIGS. 17A and 17B are exploded and perspective views of the needle in the needle module of FIGS. 11 and 12, respectively, illustrating the assembly of the needle;

FIGS. 18A and 18B are perspective views of the cap in the needle module of FIGS. 11 and 12;

FIG. 19A is a perspective view of another needle module, according to an embodiment of the present disclosure;

FIG. 19B is a side elevation view of the needle module of FIG. 19A;

FIG. 19C is an enlarged cross-sectional view taken along the line 19C-19C of FIG. 19A.

### DETAILED DESCRIPTION

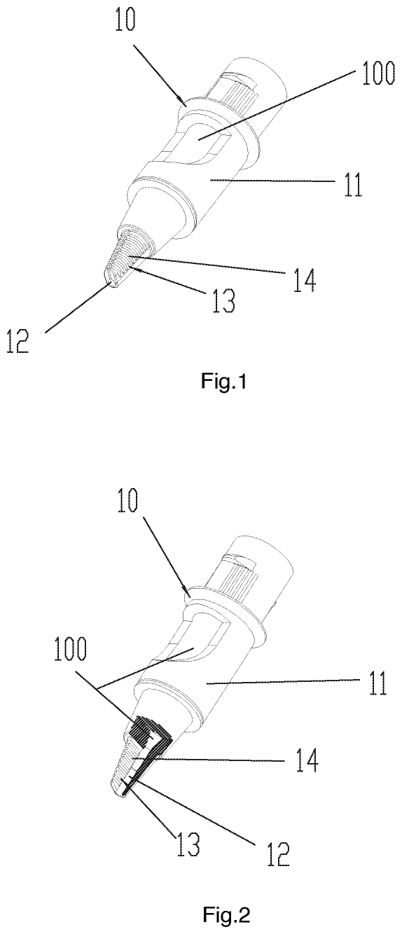

Embodiments of the present disclosure relate to tattoo devices and needle assemblies as illustrated in FIGS. 1 to 19C.

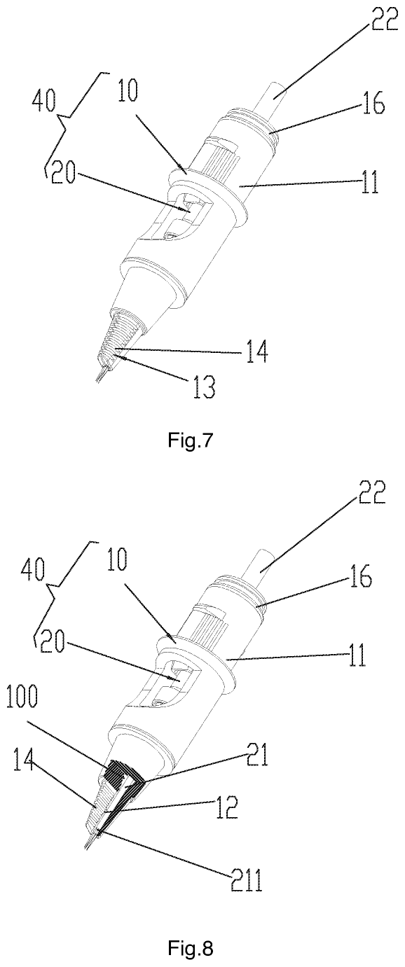

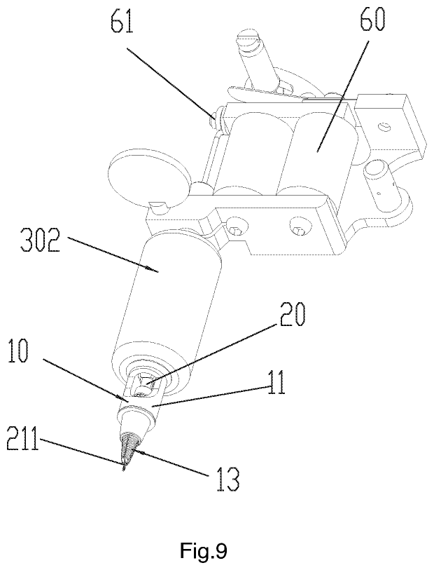

As shown in FIGS. 1-2, a tattoo needle tip 10 with a capillary ink reservoir according to one embodiment of the present disclosure includes a needle tip body 11. A needle passage 100 is provided through the needle tip body 11 for placing a tattoo needle 20 (see FIG. 7). As depicted, the needle passage includes a tubular longitudinal channel extending between a lower end and an upper end of the needle tip body 11. A needle opening 12 is provided at the lower end of the needle tip body 11. The needle opening 12 is a hole extending through the end wall of the needle tip body 11. The needle opening 12 is communicated with the needle passage 100. The sharp end 211 (see FIG. 8) of the tattoo needle can extend out from the needle opening 12. One capillary ink reservoir 13 is provided on the needle tip body 11 for maintaining and storing ink. The capillary ink reservoir 13 has at least one capillary ink containing body 14. The ink for tattooing can be maintained and stored in the capillary ink containing body 14 of the capillary ink reservoir 13 by capillary action. The capillary ink reservoir 13 is adjacent to the needle opening 12. The ink in the ink containing body 14 flows to the needle opening 12 directly and feeds to the sharp end 211 (see FIG. 9) of the tattoo needle.

The ink reservoir 13 is integrally or detachably formed at the needle tip body 11. The ink containing body 14 may be a plurality of capillary apertures. As depicted in FIGS. 1 and 2, the capillary apertures in the ink containing body 14 form grooves that extend generally transversal to the tattoo needle on the inner surface of the needle passage 100 at the needle opening 12. As depicted, the grooves may be distributed along the full longitudinal extent of the needle opening 12. The gap in the capillary apertures is a capillary gap, whereby the ink reservoir 13 can be used to absorb and retain ink by capillarity.

In different embodiments, the ink reservoir 13 may be formed of porous material, and the ink containing body 14 may have capillary pores in the ink reservoir 13 and have the function of capillarity. Alternatively, the ink reservoir 13 may be formed of fibrous material, such as a plurality of fibers, and the gaps between the fibers in the ink reservoir 13 may form the ink containing body 14 and may be capillary gaps with the function of capillarity.

Compared with the conventional cavity with smooth inner wall, the embodiment described above utilizes the capillary ink reservoir 13 to store and supply ink, which can store a large amount of ink, and does not easily cause leakage of ink. The ink reservoir 13 is located to be adjacent to the needle opening 12 with a plurality of ink containing bodies 14 being formed at the wall of the needle opening 12. The ink in the ink containing body 14 flows to the needle opening 12 directly, without a special ink guide channel between the ink containing body 14 and the needle opening 12. Compared with U.S. patent No. 20130226211 A1 and Chinese patent No. 202538157U, this embodiment of the present disclosure can improve flow of ink when ink with poor mobility and high viscosity passes through the ink guide channel. This embodiment can supply ink to the needle opening timely and sufficiently, whereby tattooists do not have to insert the needle tip into the ink cup deeply as Chinese patent No. 202538157U does for immersing the ink containing body when dipping ink. Ink with poor mobility and high viscosity is directly immersed to supply the ink containing body without passing through the ink guide channel, when tattooists are dipping ink. Thus, a tattooist may dip and add ink quickly and easily with this embodiment.

The ink reservoir 13 and the needle tip body 11 can be integrated and manufactured as one piece and connected thereon, or can be manufactured separately, then connected together.

As shown in FIGS. 1-2, in the embodiment, the needle opening 12 is in the shape of a hole, the ink reservoir 13 is adjacent to the needle opening 12, the ink containing body 14 is a capillary slot directly formed in the wall of the needle opening 12, and the ink in the ink containing body 14 flows to the needle opening 12 directly.

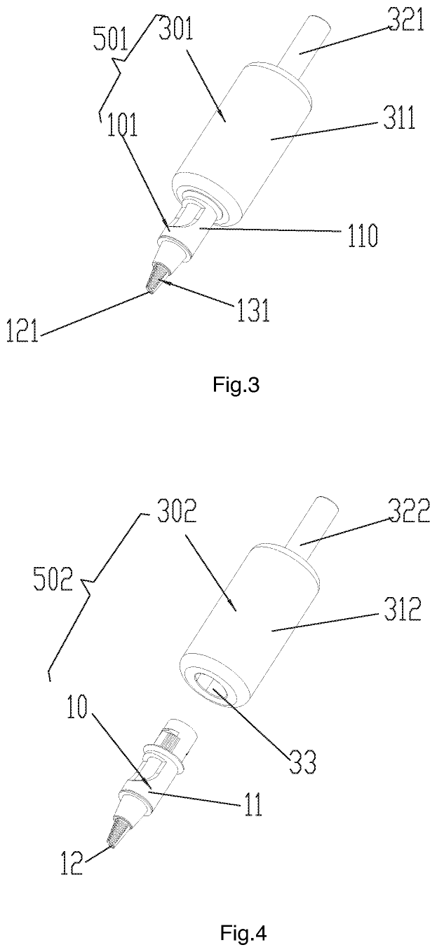

Please refer to FIG. 3, a combined device 501 according to the present disclosure includes a handle 301 and a tattoo needle tip 101 assembled to the handle 301. The structure of the tattoo needle tip 101 is similar to the abovementioned tattoo needle tip 10 with the capillary ink reservoir. In the embodiment shown in FIG. 3, the handle 301 and the tattoo needle tip 101 with the capillary ink reservoir are integrated and manufactured as one piece. The combined device forms a combined component of a tattoo device, tattooists can install a tattoo needle on the combined device, and then install them to a tattoo machine for use.

As shown in FIGS. 1-3, the tattoo needle tip 101 with the capillary ink reservoir is connected to the handle 301 integrally. The handle 301 includes a holding body 311 and a handle neck 321 located at one end of the holding body 311. One end of the needle tip body 110 distant from the needle opening 121 is integrally connected with the other end of the holding body 311. Furthermore, the tattoo needle tip 101 includes the ink reservoir 131 having the ink containing body for maintaining and storing ink. A guide hole (not shown) is defined in the handle 301. The guide hole extends through the handle neck 321 and the interior of the holding body 311 successively, and is in communication with the needle passage inside the tattoo needle tip 101.

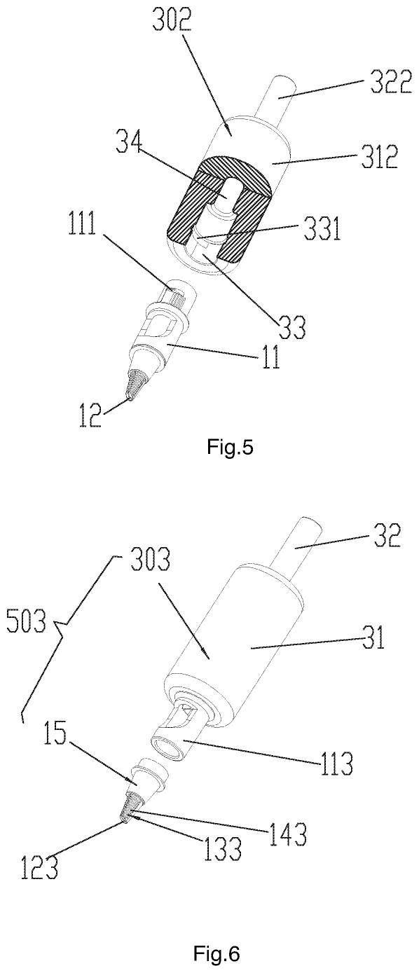

As shown in FIGS. 4-5, in combination with FIGS. 1-2, a combined device 502 according to another embodiment of the present disclosure is shown. A tattoo needle tip 10 with the capillary ink reservoir is installed at the handle 302 detachably. The handle 302 includes a holding body 312, a handle neck 322 located at one end of the holding body 312, and a needle tip installation portion 33 located at the other end of the holding body 312. A guide hole 34 is defined to extend through the handle neck 322 and the holding body 312 successively, thereafter to communicate with the needle tip installation portion 33. The tattoo needle tip 10 with the capillary ink reservoir can be detachably installed to the needle tip installation portion 33. In the embodiment, the needle tip installation portion 33 is located in the guide hole at one end of the holding body 312. The needle tip installation portion 33 has a containing slot 331. Corresponding to the containing slot 331, a snap-fit portion 111 is positioned on the outside of one end of the needle tip body 11 distant from the needle opening 12. The snap-fit portion 111 of the needle tip body 11 is snap-fitted in the containing slot 331, which forms a detachable locking mechanism of snap-fit style, so as to install the tattoo needle tip 10 to the handle 302 detachably. The needle tip body 11 and the needle tip installation portion 33 also can be installed by means of engagement of an axis and a hole, and so on. Instead of changing the handle, the detachable installation between the tattoo needle tip 10 and the handle 302 is convenient for tattooists to change tattoo needles of different specifications and needle tips adapted for tattoo needles of different specifications when tattooing.

Referring to FIG. 6, a combined device 503 according to a further embodiment of the present disclosure includes a needle tip casing 15 and the handle 303. The needle tip casing 15 is installed to the handle 303 detachably. Meanwhile, in combination with FIGS. 1-2, the structure of the needle tip casing 15 and the handle 303 can be understood. In FIGS. 1-2, the capillary ink reservoir 13 and the tattoo needle opening 12 are integrated and manufactured as one piece, which forms the needle tip casing 15 shown in FIG. 6. Similar to the structure shown in FIGS. 1-2, the needle tip casing 15 includes an ink reservoir 133 for maintaining and storing ink. At least one capillary ink containing body 143 is provided in the ink reservoir 133. The ink for tattooing can be maintained and stored in the ink containing body 143 of the ink reservoir 133, and the ink reservoir 133 is adjacent to the needle opening 123. The needle tip casing 15 is detachably installed to the needle tip body 113, and the needle tip body 113 may be integrally or detachably connected to the handle 303. The structure of the needle tip body 113 can be with reference to the needle tip body 11 shown in FIGS. 1-2. Users may install needle tip casings 15 adapted for tattoo needles of different specifications.

As shown in FIGS. 7-8, in combination with FIGS. 1-2, a combined device 40 in accordance with a further embodiment of the present disclosure includes a tattoo needle tip 10 and a tattoo needle 20 installed in the tattoo needle tip 10.

As shown in FIGS. 1-2, the tattoo needle tip 10 includes a needle tip body 11. A needle passage 100 is defined inside the needle tip body 11. A needle opening 12 is defined at one end of the needle tip body 11 and extends through the end wall of the needle tip body 11. The needle opening 12 is communicated with the needle passage 100. An ink reservoir 13 is provided on the needle tip body 11 for maintaining and storing ink. At least one capillary ink containing body 14 is provided on the ink reservoir 13. The tattoo needle 20 includes a needle head 21 and a needle bar 22 connected with the needle head 21. Most of the tattoo needle 20 is contained in the needle passage 100 of the needle tip body 11, and the sharp end 211 of the needle head 21 is located in the needle opening 12 when the sharp end 211 dips ink. In order to connect to an element driving the tattoo needle 20, the needle bar 22 extends out of the needle passage 100 from one end of the needle tip body 11 distant from the needle opening 12. The ink for tattooing can be maintained and stored in the ink containing body 14 of the ink reservoir 13, and the ink reservoir 13 is adjacent to the needle opening 12. A plurality of ink containing bodies 14 are formed in the wall of the needle opening 12. The ink in the ink containing body 14 will directly flow to the needle opening 12 for feeding the sharp end 211 of the tattoo needle 20 to continue tattooing.

In the embodiment, the other end of the needle tip body 11 opposite to the needle opening 12 is an open end. Furthermore, a cover 16 is covered on the open end. A hole is defined in the cover 16 for the needle bar 22 of the tattoo needle 20 to extend out.

Referring to FIG. 9, and combining with FIGS. 1-8, a combined device according to a further embodiment of the present disclosure includes a tattoo needle tip 10, a handle 302, a tattoo needle 20 and a tattoo machine 60. When in use, assemble the aforesaid tattoo needle tip 10 with the capillary ink reservoir, the tattoo needle 20 and the handle 302 together, connect the handle 302 to the tattoo machine 60, and insert the needle bar 22 of the tattoo needle 20 into the guide hole of the handle 302 to connect to a driving element 61 connected to the tattoo machine 60. Under the driving of the tattoo machine 60, the driving element 61 drives the tattoo needle 20 to do a straight-line reciprocating motion in high frequency in the tattoo needle tip 10, therefore, tattooists control the tattooing of the tattoo needle 20 by holding the handle 302. The capillary ink reservoir 13 of the tattoo needle tip 10 is adjacent to the needle opening 12, and the ink containing body 14 of the ink reservoir 13 is connected to the needle opening 12 directly. During operation, the tattoo needle 20 is doing a straight-line reciprocating motion in high frequency. The sharp end 211 of the needle wetted by ink rushes out from the needle opening 12 and stabs into the skin, then the sharp end 211 of the needle returns into the needle opening 12 and rushes out again with dipped ink. When adding ink, the tattoo needle tip 10 is inserted into a small ink cup, so that the ink immerses and fills the ink containing body 14 of the ink reservoir 13. When tattooing, the ink in the needle opening 12 is continuously dipped by the sharp end 211 of the needle head 21 of the tattoo needle 20 and enters into the skin. The ink in the ink containing body 14 will be directly supplied to the needle opening 12 due to the capillary force and the motion of the tattoo needle 20. The ink stored in the tattoo needle tip 10 by dipping and adding ink once can feed to the sharp end 211 of the tattoo needle 20 for many times of dipping. As a result, tattooists can avoid dipping ink frequently when tattooing. Moreover, it is quick and easy to dip and add ink.

By locating the ink reservoir at the needle tip, the above disclosed embodiment utilizes the capillary principle to store ink in the ink reservoir, which can store and retain a great amount of ink. Further, the ink storage grooves at the inner surface of the needle opening are extending transversely and the sharp end of the tattoo needle moves longitudinally across the ink storage grooves. Therefore, the leakage of ink is not easy to happen. The ink containing body of the ink reservoir is connected to the needle opening directly, and the sharp end of the needle continuously dips the ink in the needle opening via the reciprocating motion of the tattoo needle, realizing that dipping and adding ink once, the tattoo needle tip keeps tattooing for a longer time, so as to avoid dipping and adding ink frequently when tattooists are tattooing. Moreover, it is quick and easy to dip and add ink to the ink reservoir.

Although the tattoo needle tip described above and illustrated in FIGS. 1-9 are convenient to use in some applications, tests have shown that in some cases drawing of the ink from the ink storage grooves may be interrupted even when there is still substantial ink in the storage grooves.

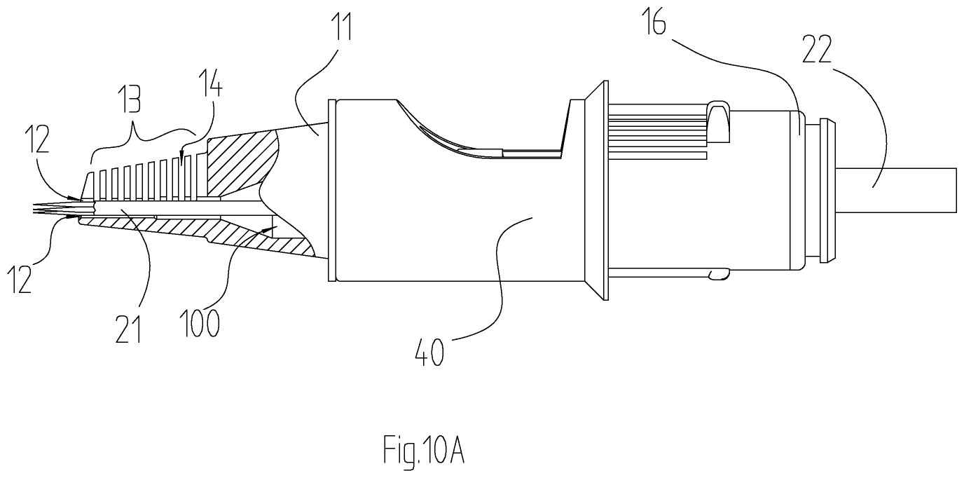

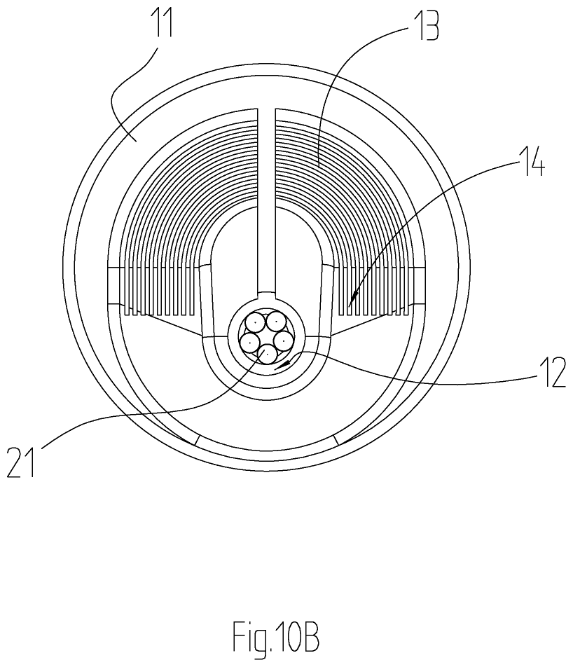

It has been recognized that, as illustrated in FIGS. 10A to 10C, depending on the needle or needle bundles used in the particularly application, the gap between the tattoo needle sharp end and the inner surface of the needle passage or opening 12 may be too big to continuously draw ink out of the ink storage grooves and maintain continuous and efficient ink flow from the ink storage grooves to the needle opening 12 and tattoo needle.

More specifically, as illustrated in FIGS. 10A and 10B, when the needle head 21 is centrally positioned in the needle opening 12, the gap between the needle head 21 and the inner surface of needle opening 12 is generally uniform. For a given size or diameter of the needle opening 12, this gap can vary depending on the size(s) of the individual needles, the number of needles used, the arrangement and spacing between the needles, and possibly other factors. For example, during manufacture a plurality of needles may be welded or soldered together to form the needle head 21. As better depicted in FIG. 10B, five needles are included in the example needle bundle shown, for illustration purposes. The sizes of the individual needles and the packing of the needles and their overall size may vary due to different manufacturing or engineering tolerances and different arrangements of the needles in the bundle. In some cases, the amount of soldering or welding materials left between the needles may vary due to various reasons. These variations can result in different diameters or overall sizes of the needle head 21, and variations in the gap between the needle head 21 and the inner surface of needle opening 12.

In addition, the gap between the needle head 21 and the inner surface of needle opening 12 will become smaller if more needles, such as seven or eight needles, of the same needle size are included in the needle bundle. The gap will become larger if fewer needles, such as four or three needles, of the same needle size are included in the needle bundle. If each needle has a larger diameter, even for the same number of needles, the gap will become smaller. If each needle has a smaller diameter, even for the same number of needles, the gap will become larger. During manufacture and operation, the user may prefer to be able to adjust the number of needles and the needle sizes without changing the needle tip body or other parts of the needle assembly. As a result, the gap between the needle and the inner surface or the grooves may vary, which in turn affects the ink drawing performance. When the gap is too large, even if there is still some capillary action, the ink drawing effect may be too small or weak to maintain continuous and efficient ink drawing from the grooves even when there is still plenty of ink left in the grooves.

The situation may become worse if the needle head 21 is being pushed away from the grooves as illustrated in FIGS. 10C and 10D, where the needle head 21 is in contact with the inside of the needle opening 12 that is opposite to ink containing body 14 of the ink reservoir 13. In this case, the gap between the needle head 21 and the ink reservoir becomes larger and capillary drawing of the ink from the ink reservoir 13 is reduced or even terminated. Such a situation may occur during operation when the operator presses the tattoo needle against the skin and moves in different directions. Such a situation may also occur if the needle head 21 is biased by something in the needle module for other reasons.

To prevent such inefficient usage of the stored ink, in an improved embodiment, a biasing member may be provided to bias the tattoo needle or needle head towards the inner side of the needle opening where the ink storage grooves are located or nearby to reduce the capillary gap between the sharp end of the tattoo needle and the grooves, and to maintain a smaller capillary gap during operation, so as to continuously draw ink out of the grooves by improved capillary action. As can be understood, a smaller capillary gap can provide improved and more reliable capillary action to improve drawing of the ink from the ink storing grooves.

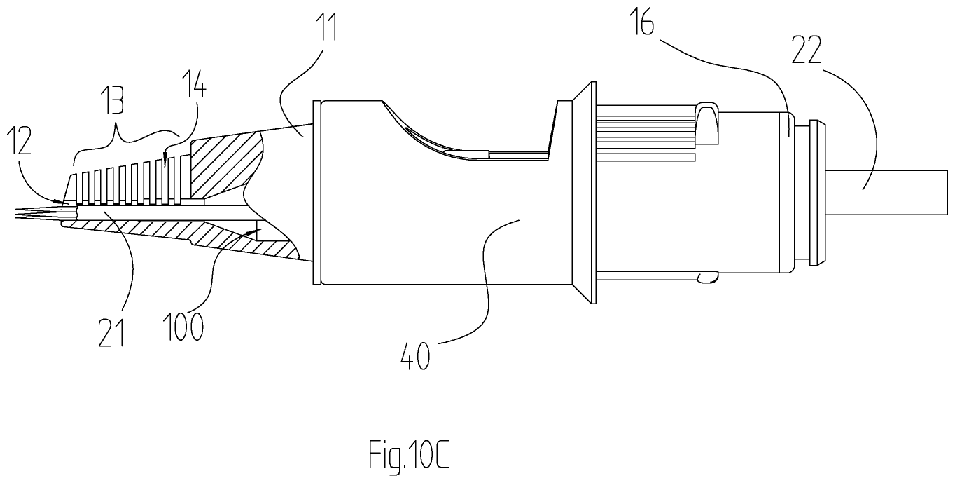

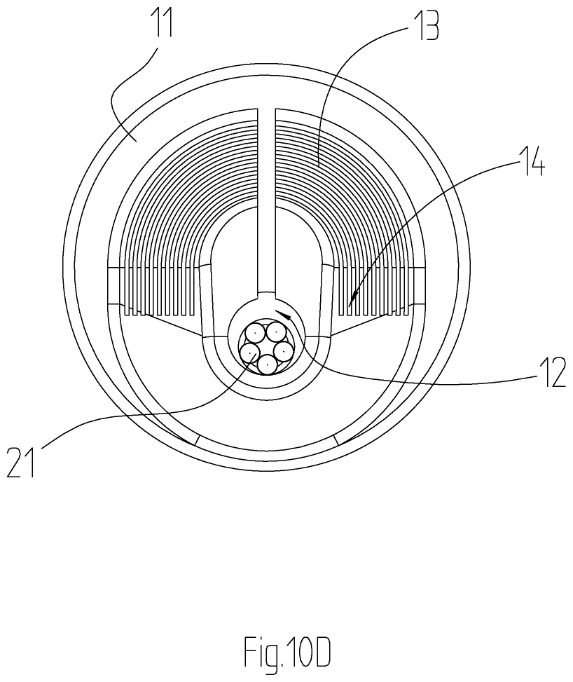

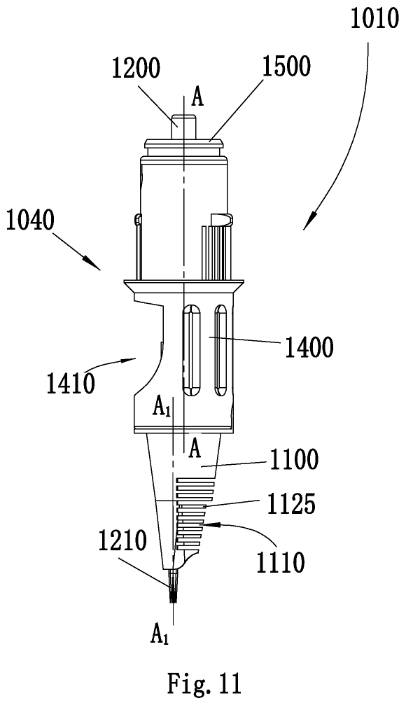

For example, a specific embodiment of such a device is provided in a needle module 1010 as illustrated in FIGS. 11 to 14C.

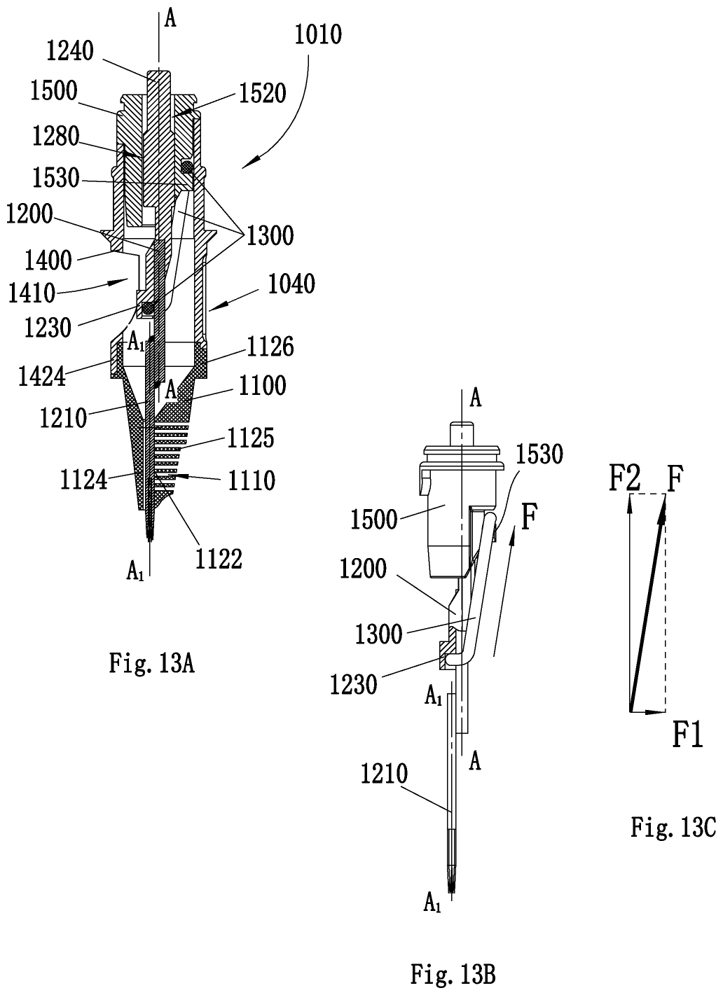

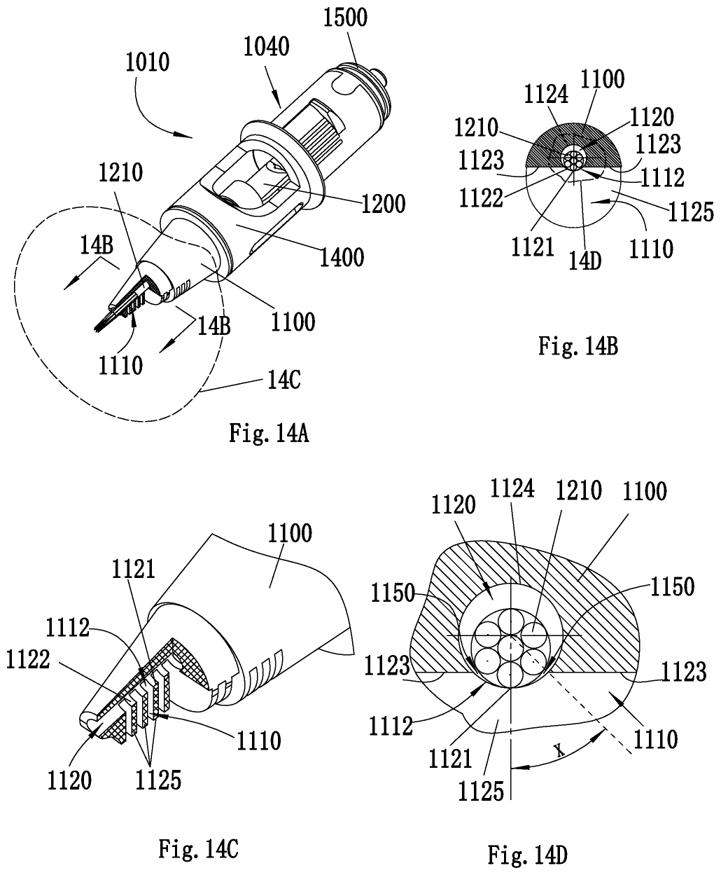

As depicted in FIGS. 11 to 13A, needle module 1010 includes a needle housing 1040 for movably mounting a needle bundle 1200 therein. As can be appreciated, the needle housing 1040 can be considered to be a needle tip body. The needle housing 1040 is formed by a tubular body portion 1400, a mouthpiece 1100 and a cap 1500. Housing 1040 has a tubular longitudinal channel 1420 therein. The channel 1420 has an upper open end 1422 and a lower open end 1424. A mouthpiece 1100 with an opening 1120 is provided at the lower end 1424 and a cap 1500 is provided at the upper end 1422. Mouthpiece 1100 has a guide surface 1122 (see FIG. 13A) for guiding movement of the needle and feeding ink to the needle, as will be detailed below. It is sufficient to note here that the guide surface has ink storing grooves thereon and the grooves are positioned on the opposite side as compared to the ink reservoir 13 on the needle tip body 10 shown in FIGS. 1-10D, as can be best seen by comparing FIG. 10C and FIG. 13A, and comparing FIG. 10D and FIG. 14D. The channel 1420 may be formed with a tubular body portion 1400, which may have an observation window 1410. Channel 1420 has a longitudinal axis denoted as axis A in the figures. The opening 1120 in the mouthpiece 1100 has a longitudinal axis denoted as axis A₁ in the figures.

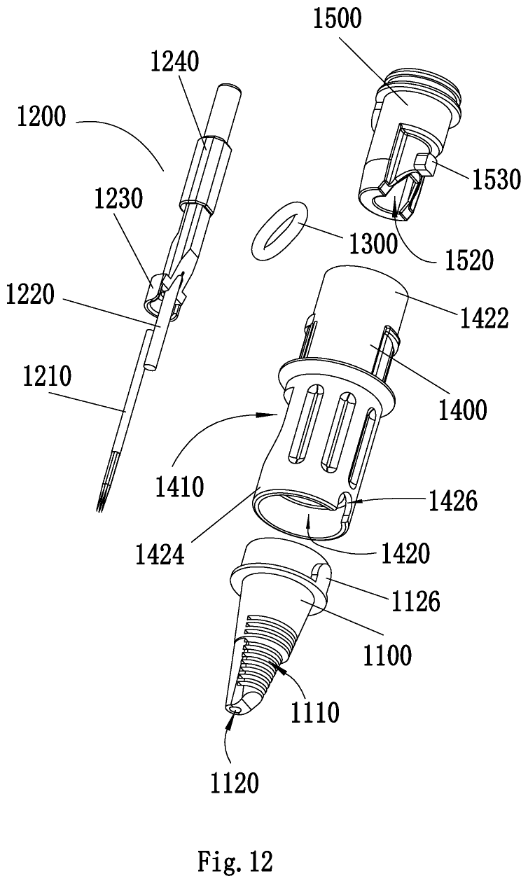

Mouthpiece 1100 and cap 1500 may be connected and engaged with the lower and upper ends of body portion 1400 in any suitable manner, with any suitable engagement or locking mechanism. For example, these parts may be engaged by tabs, threads, clamps, pins, keys, and corresponding openings, notches, threads, holes, keyways, or the like as can be understood by those skilled in the art. As depicted, both mouthpiece 1100 and cap 1500 may frictionally engage the inner walls of body portion 1400, and are interlocked in the mounted positions with a tab. For example, as illustrated in FIG. 12, the housing body portion 1400 may include a mating notch 1426 at the lower end 1424, and the mouthpiece 1100 may include a corresponding mating stab 1126 that matches and mates with the mating notch 1426 for orienting and positioning the mouthpiece 1100 in relation to the housing body portion 1400.

As will be appreciated and as in conventional needle modules, needle bundle 1200 is housed and guided in housing 1040 in a manner to allow the needle bundle 1200 to reciprocally move up and down during use. The detailed construction and mechanism for such mounting and reciprocal movement are not the focus of this disclosure, and can be implemented by a skilled person in the art according to known techniques or constructions, except in aspects specifically described below. Thus, some of these details will not be discussed herein.

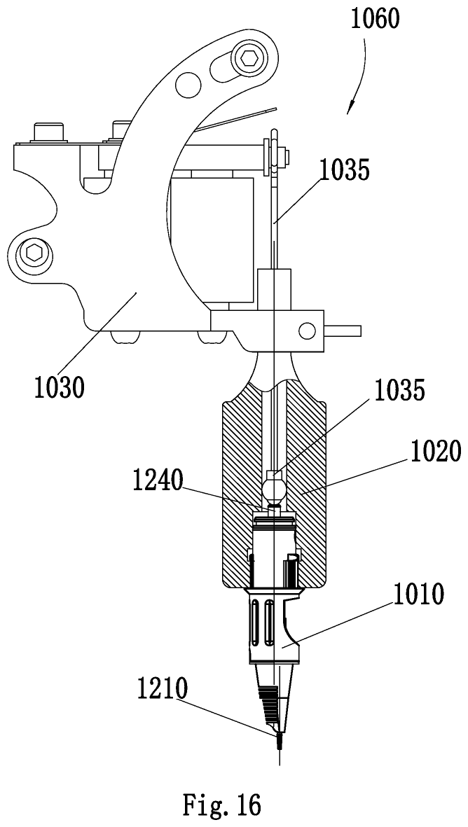

It is noted that, however, to facilitate the reciprocal movement of the needle bundle 1200, a biasing member such as a resilient band 1300 is also provided and configured to pull the needle bundle 1200 up during each movement cycle after the needle bundle 1200 is pushed down by an actuating or driving mechanism such as a drive shaft (not shown but see FIG. 16 to be discussed later). In an embodiment in the present enclosure, the biasing member such as band 1300 is not only used to facilitate reciprocal movement of the needle bundle 1200, but also used to bias the needle bundle towards the guide surface 1122 to maintain contact or a smaller capillary gap between the needle bundle 1200 and the stored ink in the ink reservoir as will be made clearer below.

As will be understood, needle module 1010 can also be considered as a needle assembly. While the depicted device is referred to as a needle module, it is only used to illustrate an embodiment of possible needle assemblies. In different embodiments, an embodiment of the present disclosure may be used in any number of different types of needle assemblies.

Returning to FIGS. 11 and 12, as depicted, needle bundle 1200 includes a tip portion 1210 (also referred to as the sharp end of the needle), a needle shaft portion 1220, a hook structure 1230, and a shaft 1240.

Tip portion 1210 may include one or more sharp pricking needles, which may be welded or soldered together, or otherwise bounded together. The pricking needles may be formed of stainless steel or any other suitable material. The individual pricking needle may have any suitable or known needle sharp end shape. The pricking needles may be arranged to form a tip portion that has a generally or substantially cylindrical or conical profile. Alternatively, the pricking needles may be arranged side-by-side to form a tip portion that has a generally flattened or band-shaped profile. Rows of side-by-side needles may also be stacked. Such different arrangements of tattoo needles are known in the art and may be referred to as “Round Liner” needles, “Round Shader” needles, “Flat” Needles, or “Magnum” needles, respectively. Tip portion 1210 may include 1-18 individual pricking needles for “Round Liner” needles and “Round Shader” needles, or may include 4-27 pricking needles for “Flat” needles and “Magnum” needles.

As can be appreciated, the cross-sectional sizes or diameters of the needles or needle bundles will affect how the ink will flow. Typically, the smaller the pricking needle size or narrower the diameter of the pricking needle, the finer and more controlled the stream of ink that flows off each pricking needle. Typically, the pricking needles in the same needle bundle may be of the same or similar sizes. The size of the pricking needles may be selected based on the desired effects by the operator or user. Different sizes may be used for different reasons. Standard sizes of pricking needles may be used. The diameters of the individual pricking needles may be 0.25 mm, 0.30 mm, or 0.35 mm in some embodiments. The designs of the pricking needles may be selected and vary as known in the art based on the desired tattooing techniques and purposes to be applied.

The number of pricking needles in a needle bundle may vary from 1 to 27 or more as desired. For example, commercially available round needle bundles typically have 1, 3, 5, 7, 8, 9, 11, 14, or 18 pricking needles in each bundle. It would also be appreciated that the overall profile of the needle bundle may change and vary depending on the number of pricking needles in the bundle, their arrangement, the amount of soldering material used, or other factors.

The pricking needles in a bundle may be welded together, such as by lead-free soldering.

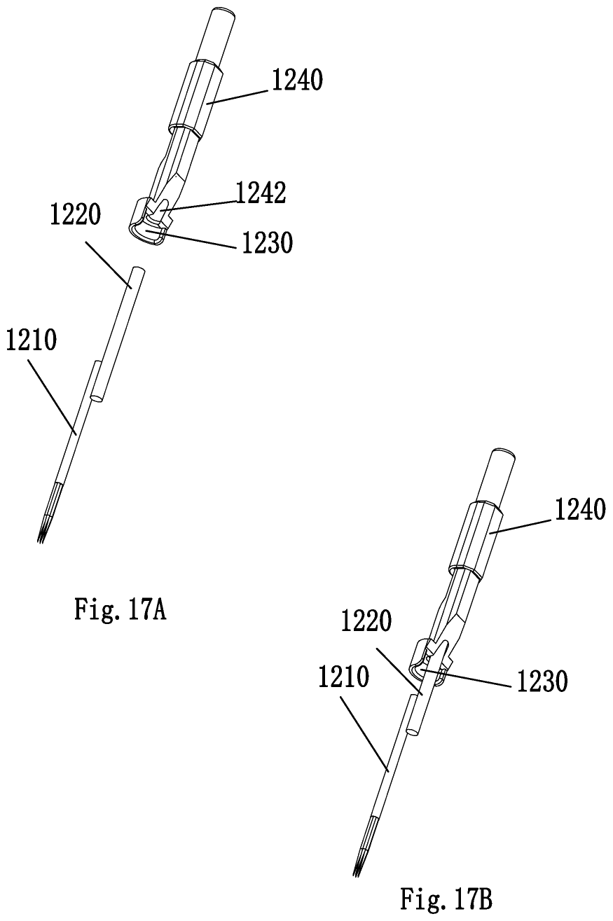

As can be seen in FIG. 12, FIGS. 13A and 13B, and FIGS. 17A and 17B, the welded pricking needles in tip portion 1210 are supported on needle shaft portion 1220. The axis of needle shaft portion 1220 and the axis of shaft 1240 are aligned with the axis A of the channel 1420 in the housing body portion 1400, but are off-set from axis A₁ of the opening 1120 of the mouthpiece 1100 and the axis of needle tip portion 1210. The needle shaft portion 1220 may be formed of stainless steel or another suitable material. It should be sufficiently rigid and strong to support stable movement of the needle tip portion 1210 during operation. Tip portion 1210 may be welded onto needle shaft portion 1220.

Needle shaft portion 1220 is joined with shaft 1240 for driving the tip portion 1210. Shaft 1240 may be formed of a plastic material or another suitable material for transmitting the axial driving force to needle shaft portion 1220 and then indirectly to tip portion 1210. As depicted in FIGS. 17A and 17B, shaft 1240 may include a cylindrical bore 1242 for receiving the needle shaft portion 1220 at the lower end of the shaft 1240. Needle shaft portion 1220 may be inserted into the bore 1242 and may be attached to the wall of the bore 1242 of the shaft 1240 with an adhesive, such as glue. At the bottom end of the shaft 1240, a hood-shaped hook 1230 is provided adjacent to the bore opening of bore 1242. The hook 1230 is shaped and sized to accommodate and retain a section of the elastic band 1300, which is looped around needle shaft portion 1220 during assembly and is retained between needle shaft portion 1220 and hook 1230. As can be appreciated, other forms or structures may also be used to retain band 1300. The hood-shaped hook 1230 provides a secure retaining mechanism for retaining the band 1300 when needle bundle 1200 moves up and down, as it prevents accidental release of the band section over the hook 1230 due to fast relative movement of the parts.

Hook 1230 may be replaced by a hook mounted on shaft 1240 in a different embodiment.

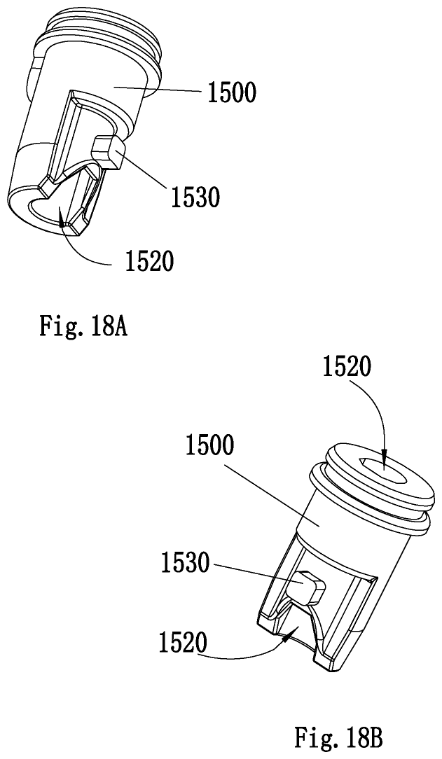

The cap 1500 may be made of a plastic material, or any other suitable material. As better seen in FIGS. 18A and 18B, cap 1500 has a through opening 1520 that allows the shaft 1240 to axially move therethrough. A stub 1530 is provided on cap 1500, which stub faces the inner wall of in the body portion 1400 of the housing 1040.

As illustrated in FIGS. 13A and 13B, a section of the band 1300 may be looped around the stub 1530 on the cap 1500, and retained between the stub 1530 and the inner wall of the body portion 1400 of the housing 1040.

An elastic looping band 1300 is provided, which, when the needle module 1010 is assembled, is looped around at one end hook 1230 and at the other end around stub 1530, so that band 1300 is hooked by hook 1230 and stub 1530 so that band 1300 is retained in place but can expand and contract when the needle bundle 1200 moves up and down during operation. The hook 1230 and stub 1530 are configured and oriented during assembly such that when band 1300 engages both hook 1230 and stub 1530, band 1300 biases the needle bundle 1200 upward and biases the tip portion 1210 against the guide surface 1122 in the mouthpiece 1100. Band 1300 may be formed of a silicone band, or a band formed of another resilient material such as latex or rubber. Band 1300 may be in the shape of an O-ring when in a relaxed state.

As better illustrated in FIGS. 13A to 13C, the band 1300 is normally positioned and oriented at a slight angle relative to the axial direction of shaft 1240, in the F direction as shown in FIG. 13B. For easier viewing, the mouthpiece 1100 and body portion 1400 are not shown in FIG. 13B. The band 1300 is normally slightly tensioned to bias the needle bundle 1200 upward (i.e. to retract) and bias the tip portion 1210 against the guide surface 1122. When the needle bundle 1200 is pushed downward by a drive shaft (not shown) through shaft 1240, the band 1300 is further stretched and provides an increased axial biasing force upward (component F2 as illustrated in FIG. 13C). However, the radial component of the biasing force (F1 shown in FIG. 13C) against the guide surface 1122 will not change significantly during axial movement of the needle bundle.

As can be appreciated, the inner diameter of the through opening 1520 in cap 1500 may be sized to be larger than the outer diameter of the shaft 1240, and there may be gap 280 between the shaft 1240 and the inner wall that defines opening 1520. Such a gap may allow smooth movement of the needle bundle 1200 and shaft 1240 in the housing 1040, reducing or preventing the risk of jamming or too much friction. Such a gap also allows the shaft 1240 and needle bundle 1200 to lean at a slight angle off the axial direction (axis-A).

Under the biasing force (denoted by F1 as shown in FIG. 13C) exerted by the band 1300, the tip portion 1210 at the bottom end of needle bundle 1200 is pressed towards guide surface 1122. A slight lean of needle bundle 1200 off the axial direction of the axis A or A₁ would reduce the stress and bending of needle bundle 1200.

As depicted, the axis A₁ of opening 1120 and the needle tip portion 1210 is offset from the axis A of the shaft 1240 and needle shaft portion 1220. The mouthpiece 1100, needle shaft portion 1220, shaft 1240, body portion 1400 and cap 1500 may be co-axial in some embodiments, as depicted in FIG. 13A (aligned along axis A). As depicted in FIGS. 11 and 13A, the axis A₁ is offset from the axis A towards the observation window 1410, so that the axis A₁ is closer to the observation window 1410 than the axis A. The distance between axis A and A₁ may be from about 1.2 mm to about 1.8 mm. The needle module 1010 is typically held by the operator so that the observation window 1410 is facing the operator during operation. As depicted, when the needle module 1010 is properly assembled, the observation window 1410 and the guide surface 1122 are on opposite sides of the axis A₁.

In different embodiments, the shaft 1240 and needle shaft portion 1220 may also be offset and non-coaxial. In such cases, the offset distance between the axis A₁ and the axis A may be even larger, such as about 2 mm to about 3 mm, or up to 5 mm.

The mouthpiece 1100 is configured to support and allow axial movement of the needle tip portion 1210 through the opening 1120. The mouthpiece 1100 may be made of a plastic material or another suitable material. The opening 1120 may have a shape configured to match the profile of the tip portion 1210 of needle bundle 1200. For example, for round or circular tip portion 1210, the opening 1120 may have a circular or diamond shape; for flat tip portion 1210, the opening 1120 may have a rectangular shape.

As depicted in the drawings, the mouthpiece 1100 and housing body portion 1400 may be generally co-axial (along axis A) but the axis A₁ of the opening 1120 may be offset from axis A of channel 1420. As can be appreciated, when the axis A₁ of the opening 1120 is offset from the axis A of the channel 1420, the needle tip portion 1210 is also offset from axis A to align with the opening 1120. Conveniently, the embodiment as depicted allows the operator to better see the position of the sharp needle end(s) during operation. To allow improved observation, mouthpiece 1100 may be made of a transparent material, such as a transparent plastic. The offset of the opening 1120 with respect to the channel 1420 also provides more space to accommodate larger ink storage grooves 1110, thus providing higher storage capacity.

The size of opening 1120 is also selected to accommodate the size of the tip portion 1210. To avoid jamming during use and to accommodate different needle sizes or needle bundle sizes, the size of the opening 1120 of the mouthpiece 1100 may be selected to allow some play of the largest needle bundle to be used. That is, the size of opening 1120 is slightly larger than the size of the needle tip portion 1210, so that there is a gap between the inner wall of opening 1120 and the needle tip portion 1210 on the opposite side of the guide surface 1122. This gap allows smooth movement of the needle tip portion 1210 and size variations of the tip portion 1210, without jamming.

Even though there is a gap between the needle tip portion 1210 and the opening 1120, the risk that the tip portion 1210 will vibrate sideways is low as the tip portion 1210 is biased against the guide surface 1122, which provides a stable support surface for the movement of the tip portion 1210. In other words, even when the size of the opening 1120 is relatively large, and there is a gap between the tip portion 1210 of the needle bundle 1200 and the inner surfaces of the mouthpiece 1100, the biasing force F1 causes the tip portion 1210 to contact and abut against the guide surface 1122, and maintain the contact during operation (reciprocal movement of the needle bundle 1200).

Mouthpiece 1100 is also configured to function as an ink feeding device. Specifically, as illustrated in FIG. 13A, and in FIGS. 14A to 14D, ink storage grooves 1110 defined by groove walls 1125 are provided on guide surface 1122, for storing ink. Each groove 1110 extends generally transversely in relation to the axial direction and the direction of movement of the needle bundle 1200. Groove walls 1125 and grooves 1110 may be generally parallel to one another, and are located near, but above, the lower open end of mouthpiece 1100. Grooves 1110 may extend radially to the external surface of the mouthpiece 1100 to allow easy filling of ink during use. That is, each groove 1110 may be open to the external surface of mouthpiece 1100 as depicted in these figures. Each individual groove 1110 is formed between two opposing groove walls 1125 having a thin gap between the opposing groove walls 1125 such that when the mouthpiece 1100 is brought into contact with an ink source (such as when it is dipped in an ink bottle), ink can be sucked into and fill the gap due to capillary action. The ink in the gap can be normally retained in place due to surface tension and adhesion of the ink liquid to the groove walls 1125.

However, when the tip portion 1210 of the needle bundle 1200 moves downward across the grooves 1110 while being pressed against the guide surface 1122, the moving needles will contact the liquid surface of the ink stored in the grooves 1110 and bring ink out of grooves 1110, and then carry the ink with the needles or allow the ink to flow along the needles and be applied to the skin. Close contact with the guide surface 1122 also facilitates flow of the ink into narrow gaps 1150 (see FIG. 14D) that exist between the needles in the needle bundle 1200 and the guide surface 1122.

For example, as better illustrated in FIGS. 14B and 14D (also see FIG. 14C), in an example bundle of seven (7) needles, it is possible that only the bottom needle is in contact with the guide surface 1122, at a point 1121. A section of the bottom needle may be in contact with the guide surface 1122 between the grooves 1110, and may extend across the grooves in a direction that is particular to the traversal directions along which the grooves 1110 extend. However, narrow gaps 1150 also exist between the curved guide surface 1122 and some of the needles in tip portion 1210 of the needle bundle 1200. A much larger gap is present between the tip portion 1210 and the opposite wall surface 1124 of the inner channel of the mouthpiece 1100 as depicted in FIG. 14B (also see FIG. 14D).

The grooves 1110 are also open at the guide surface 1122, with ink contacting openings 1112. The width of each ink contacting opening 1112 may be about 0.3 mm to 0.6 mm. The width may vary and may be selected based on the ink fluid properties. In a particular embodiment, the width of opening 1112 may be about 0.4 mm.

The number of grooves 1110 may also vary and may be selected based on a number of factors, such as the amount of ink to be applied and the size and length of the needles used, and production or manufacturing considerations. In some embodiments, there may be 3 to 12 grooves, such as 5 to 10 grooves.

The contact opening 1112 of each groove 1110 at the guide surface 1122 may extend circumferentially by a selected angle θ around the axial direction (see axis A₁ in FIG. 11). The contact opening 1112 may extend symmetrically about the contact point 1121, by an angle of X on each side (see FIG. 14D), where θ=2X. In different embodiments, angle θ may vary from about 120° to about 360°. For example, angle θ may be about 120°, 140°, 160°, 180°, or 200°.

The external openings of grooves 1110 may extend circumferentially at similar angles or larger angles. As depicted in FIGS. 14B and 14D, in an embodiment, the external openings of grooves 1110 may extend continuously, and may be limited/defined by a wall 1123 that extends along the line between the ends of the contact opening 1112.

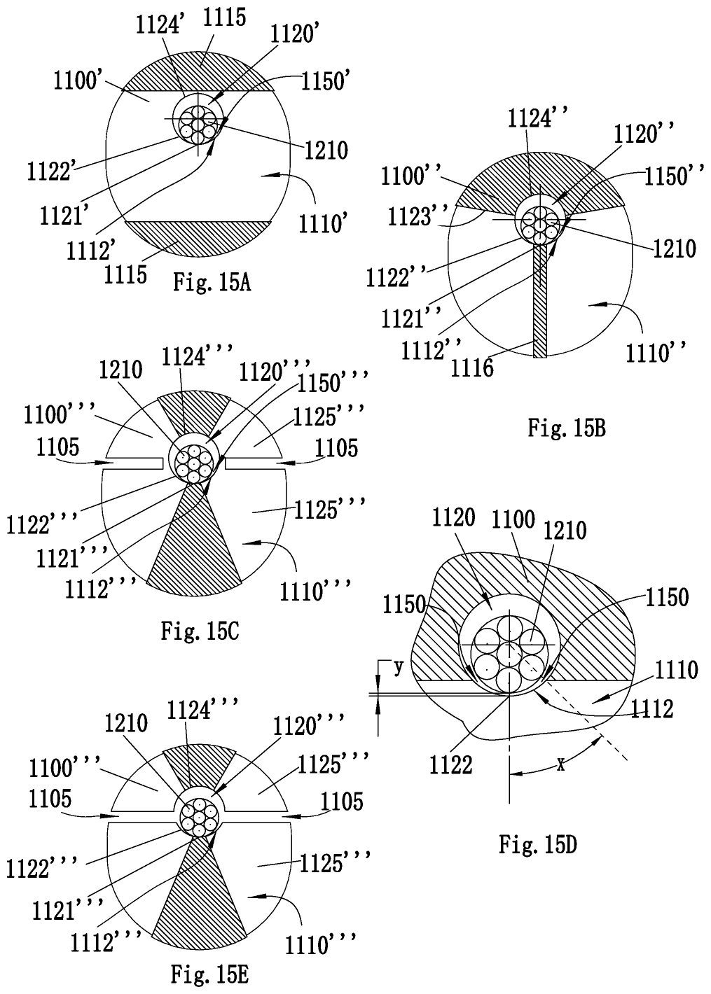

In alternative embodiments, the external openings of grooves 1110 may be segmented, as illustrated in FIGS. 15A, 15B and 15C.

FIG. 15A illustrates a portion of a variant of the mouthpiece 1100′ having ink storage groove 1110′ with contact opening 1112′, the angle θ is 360°, an opening 1120′, a guide surface 1122′, and an opposite surface 124′. Needle bundle tip portion 1210 is received in opening 1120′, and contacts the guide surface 1122′ at contact point 1121′. There is a narrow gap 1150′ between tip portion 1210 and the guide surface 1122′ around the contact point 1121′. In FIG. 15A, the external opening is separated by solid sections 1115. Sections 1115 provided at opposite sides can provide reinforcement of the structural integrity. It is possible to have a section 1115 at only one side, in which case, however, the mechanical strength of the structure may be reduced and the mouthpiece 1100 may be easier to bend or deform.

FIG. 15B illustrates a portion of a variant of the mouthpiece 1100″ having ink storage groove 1110″ with contact opening 1112″, an opening 1120″, a guide surface 1122″, and an opposite surface 124″. Needle bundle tip portion 1210 is received in opening 1120″, and contacts the guide surface 1122″ at contact point 1121″. There is a narrow gap 1150″ between tip portion 1210 and the guide surface 1122″ around the contact point 1121″ and at contact opening 1112″. In FIG. 15B, the external opening is separated by a reinforcing section 1116, which can increase the structural strength and prevent or reduce bending or deformation.

FIG. 15C illustrates a portion of a variant of the mouthpiece 1100′″ having ink storage groove 1110″ with contact opening 1112′″, an opening 1120′″, a guide surface 1122′″, and an opposite surface 1124′″. Needle bundle tip portion 1210 is received in opening 1120′″, and contacts the guide surface 1122′″ at contact point 1121′″. There is a narrow gap 1150′″ between tip portion 1210 and the guide surface 1122′″ around the contact point 1121′″ and at contact opening 1112′″.

In FIG. 15C, fluid conduits 1105 are provided on groove walls 1125′″ to allow fluid communication between different grooves 1110′″. Conduit 1105 thus connects grooves 1110′″. The width of conduit 1105 is smaller than the width of the groove 1110′″. For instance, if the groove 1110′″ has a width of 0.4 mm, conduit 1105 may have a width of about 0.3 mm.

In other embodiments, conduit 1105 may be deeper or shallower, and may even extend all the way to the guide surface 1122′″ so that ink may be fed directly to tip portion 1210, as illustrated in FIG. 15E.

As can be appreciated, section 1115 or 1116 may provide a stronger construction and stabilize the groove walls 1125, and thus the gaps therebetween, although providing section 1115 or 1116 may reduce the ink storage volume in the grooves 1110.

In the embodiment illustrated in FIG. 15B, the bottom needle or the tip portion 1210 of the needle bundle 1200 may be in continuous contact with the guide surface 1122″ at contact point 1121″. Section 1116 may be formed of a reinforcing material to improve the mechanical strength of the mouthpiece. The guide surface at the section 1116 may also be smooth, and may optionally be provided with special surface treatment for reduced friction between the needle(s) and the guide surface 1122″.

With the added section 1115, or 1116, it is also possible to further extend the circumferential angle of each groove 1110′ or 1110″, as illustrated in FIGS. 15A and 15B respectively, such as to an angle higher than 180° or is 360°. Groove wall 1123″ may also extend at an angle relative to the line between the ends of the contact opening 1112″, to allow larger external openings for easier ink filling and increased ink storage capacity.

As illustrated in FIG. 15D, it is possible in some embodiments that some portions of the bottom needle are not in contact with the guide surface 1122, but is very close to the guide surface 1122 due to the biasing force (F1), separated by a small distance “y”. As long as the gap y is small enough, ink may still be drawn from ink storage grooves 1110. A benefit of no-contact between the needle and the guide surface 1122 is that there is reduced or no friction.

Needle module 1010 may be pre-sterilized and packaged to keep it clean during storage and transportation.

The needle module 1010 and its parts may be manufactured from suitable materials based on technologies known to those skilled in the art. For example, various parts in the needle module 1010 may be made from corresponding materials used in conventional needle modules, such as those used in tattooing needle modules. The parts may be formed and machined using known processing techniques, as can be understood by those skilled in the art in view of the present disclosure.

During use, needle module 1010 is installed on a corresponding tattooing device. For example, as illustrated in FIG. 16, needle module 1010 may be coupled, through or by a needle handle 1020, to a base device 1030.

The base device 1030 may typically include a needle actuator with a drive shaft 1035 for actuating and driving the needle tip portion 1210 through shaft 1240.

The handle 1020 may have a generally tubular shape, and may be cylindrical.

The handle 1020 and base device 1030 may be constructed and operated as disclosed in U.S. patent application Ser. No. 15/691,125 by Xiao, the entire contents of which are incorporated herein by reference.

The assembled needle module 1010, handle 1020, and base device 1030 form an ink applicator 1060, or in particular, a tattooing device.

During operation, an operator may hold the ink applicator 1060 with handle 1020 in her hand, and dip the mouthpiece 1100 in an ink container (not shown) to fill the ink storage grooves 1110 with the desired ink. The needle actuator is activated to drive the drive shaft 1035 and consequently needle shaft 1240 and tip portion 1210 of the needle bundle 1200 downward. After each downward stroke, the resilient band 1300 is stretched and pulls the tip portion 1210 back up once the drive shaft 1035 stops the downward movement and moves upward. The needle actuator then re-starts the downward drive in the next cycle. This process repeats so as to drive the needle bundle 1200 to reciprocally move longitudinally along the axial direction of the shafts 1035 and 1240. The base device 1030 may be configured to operate at a drive frequency of about 80 to 150 Hz. The operator may adjust the operating frequency during operation. In some tattooing machines, the tattoo needles may be operated to puncture the skin from 50 to 10,000 times per minute. The needles may penetrate the skin and reach a depth of about 1 mm. The vertical moving distance of the drive shaft 1035 is typically about 2 mm to 5 mm, and the needle bundle 1200 may similarly move about 2 to 5 mm during each stroke.

In different embodiments, drive shaft 1035 may be connected to needle bundle 1200 through a coupling member (not shown). During a stroke, drive shaft 1035 may push needle bundle 1200 downward, and thus stretch the band 1300. After reaching the bottom of the stroke, the drive shaft 1035 and band 1300 will both pull needle bundle 1200 back up.

As the tip portion 1210 moves up and down, the operator may bring it to contact a subject's skin to apply ink to the skin, as in a conventional operation, which can be understood by those skilled in the art.

As discussed above, due to the biasing force (F1) applied by band 1300, tip portion 1210 is biased against the guide surface 1122 in mouthpiece 1100 while moving up and down due to the longitudinal reciprocal movement, and can draw ink out of ink storage grooves 1110.

After each operation or use, the needle module 1010 may be removed and disposed. The handle 1020 may be next removed, and may also be disposed. The operator can take off disposable gloves and clean her hands at this time before touching other parts of the ink applicator 1060.

For the next operation, a new needle module and new handle may be connected to the base device 1030, and used similarly as described above.

The needle module 1010 may be used directly after opening the needle module packaging without further cleaning, sanitization, or sterilization, and can be disposed after a single use without cleaning or any other treatment.

Conveniently, ink flow from grooves 1110 to the sharp end of the tip portion 1210 may be stable and consistent, when there is still sufficient ink in grooves 1110. Ink residue in the grooves 1110 before re-fill can be reduced, as compared to some conventional ink feeding techniques. More efficient use of the ink and ink fill is thus possible.

As the contact openings 1112 of grooves 1110 extend perpendicular to the axial direction of tip portion 1210 and its movement direction, it is unlikely the tip portion 1210 may stuck in a groove 1110. In comparison, if the ink storage groove is parallel to the needle movement direction, the needle may be more likely to get stuck in the groove.

Further, a tattoo operator tends to hold the needle in a relatively vertical orientation or an inclined orientation close to the vertical orientation. When the grooves run parallel to the needle movement direction, i.e., vertical or nearly vertical during operation, the ink in the groove may tend to flow downward due to gravity. To prevent such undesired ink flow, the vertical grooves will need to be narrower to limit the effect of gravity. In comparison, as the groove walls 1125 between the grooves 1110 are positioned horizontally or close to horizontal during operation, gravity will have less effect on the fluid flow in the grooves 1110. As a result, relatively larger grooves (or gaps between groove walls) may be used for ink feeding in an embodiment of the present disclosure, and consequently, the amount of ink stored during each fill or re-fill is increased as compared to some conventional ink feeding devices.

With perpendicular grooves, the number of grooves may also be varied, and increased as compared to vertical grooves. With vertical grooves, the number of vertical grooves is limited due to the limited diameter of the needle size (or size of the needle bundle). Tests have shown that with vertical grooves, while the grooves directly in contact with the needle tended to allow excessive ink flow, the grooves not in direct contact with the needle tended to keep excessive residual ink even when the needle was no longer delivering ink to the skin. That is, vertical grooves further away from the needle are not very effective for feeding ink. Such limitation and problems do not exist with particular grooves in an embodiment disclosed in the present disclosure.

It might have been expected that perpendicular groove orientation is not as effective as the needle would need to travel across groove edges, which might have been expected to cut off ink flow, so that it might have been expected that perpendicular groove orientation should not be used. However, tests have surprisingly shown that example embodiments as disclosed herein could deliver and feed ink more stably and consistently, and could address a number of drawbacks of conventional feeding techniques discussed herein.

For example, without being limited to a particular theory, it is expected that, while the ink flow from grooves 1110 in a vertically held needle bundle 1200 may be mainly due to capillary action (gravity effect is limited by the horizontal groove walls 1125 as discussed above), since the small gaps 1150 between the guide surface 1122 and needle tip portion 1210 may be smaller than the groove width in grooves 1110, the capillary force in the gaps 1150 may be larger than the capillary force in the grooves 1110. As such, ink will likely be drawn out of the grooves into the small gaps 1150 due to such force differential. Ink residual in the grooves 1110 may be reduced as a result.

Another convenient effect of an embodiment disclosed herein is that the size of the opening 1120 in the mouthpiece 1100 may be adjusted with more flexibility. Due to the biasing force applied to the tip portion 1210 by the band 1300, contact and alignment of the tip portion 1210 with guide surface 1122 may be more conveniently maintained without a close-fit opening 1120. With a larger opening 1120, it is possible to reduce friction between the needle tip portion 1210 and the wall surface at opening 1120.

For clarity, it is noted that “single use” may refer to use of a needle or needle module for one complete operation on a single individual subject. During this operation, different needle modules may be used to apply different ink colors or for different purposes. For example, it may be typical to use two to five different types of needles during a single operation on a subject, depending on the complexity of the design to be applied.

As can be appreciated, various modifications may be made to the example devices illustrated in the drawings.

For example, band 1300 may be formed of any suitable material with suitable strength and resiliency. Further, the O-ring and hook structure may be replaced with another biasing mechanism or structure. Other biasing members known to skilled persons in the art may also be used as long as they provide the desired biasing force to draw back the needle and bias the tip portion against the guide surface 1122. In some embodiments, separate biasing members may be used to separately provide the longitudinal biasing force F2 and the radial biasing force F1 (see FIG. 13C). However, as can be appreciated, band 1300 conveniently provides both biasing forces with a simple construction structure. Alternative biasing members may include elastic bands, springs, resilient stabs, or the like. Suitable resilient materials may include silicones, rubbers, latex, plastics, metals, or the like.

To further reduce friction and possible adverse edge effects at the edges of contact openings 1112, the edges at openings 1112 may be treated for smooth contact, and may be rounded to eliminate sharp edges. As described above, directed contact maybe limited or reduced to a small contact point. However, when the needle bundle does not directly contact the guide surface, the distance between the needle and the guide surface should be relatively small, such as below 0.15 mm, to allow the needle to draw ink out of the grooves 1110. As noted above, a further alternative to avoid or limit edge effect, is to provide a smooth guide surface at the contact point, such as illustrated in FIG. 15B and FIG. 15C.

In some embodiments, the groove walls 1125 in grooves 1110 may be provided with cavities or holes (not shown) to store more ink. Ink storage grooves may include smaller segments of openings arranged in rows and columns. Such openings may have circular, rectangular, oval or other profiles or shapes. The arrangement of the openings and grooves may be regular or irregular. The tip portion 1210 may be biased toward and to move across the arrangement of the openings so as to draw out ink by capillary action.

As the amount of stored ink may be increased, while reducing the risk of excessive residual ink or unstable ink flow, an embodiment of the needle assembly disclosed herein may allow an operator to fill the ink less frequently, and reduce the operation time.

As now can be appreciated, when guide surface in the mouthpiece is shaped to conform to an external profile of the needle bundle, the contact area between the needle bundle and the ink stored in the grooves can be increased.

As described herein, the circumferential angle refers to the angle formed by two lines from the center of a circle to two points on the circumference of the circle. It should be noted that, however, in practice, the parts in the disclosed devices may not have perfect circular shapes, and the profiles of the parts may be generally circular and the angles may be approximate.

The longitudinal channel in the housing may be considered to have a generally circular profile even if the profile is not a perfect circle. In some embodiments, the longitudinal channel may have a generally polygonal profile, which is not necessarily perfect polygon. For example, the profile may be generally rectangular or a diamond shape, but the edges may be no-linear.

In some embodiments, the ink storage grooves may have a width of about 0.3 mm to about 0.6 mm, but in other embodiments the width may be wider or narrower.

As depicted in the figures, the housing in the needle module may include a generally conical lower portion (mouthpiece) and a generally cylindrical upper portion (body portion). However, in different embodiments, the housing and any of its components may have different shapes and sizes.

The ink storage grooves may be separated and isolated by groove walls, or may be inter-connected via channels or holes through the groove walls. The groove walls may also be perforated. The connecting channels and holes may be sized for capillary action, or may have relatively larger sizes as it is not necessary that the fluid communication between the grooves through the groove walls are effected by capillary action. Interconnected grooves may allow faster and more uniform ink loading and distribution along the needle length, and hence more effective ink feeding.

As described earlier, in an embodiment, the tip portion of the needle bundle may not contact the inner surface of the mouthpiece during operation. In such cases, the housing may include a tubular longitudinal channel and an inner surface. The channel has an upper open end and a lower open end, a plurality of ink storage grooves for storing ink by capillary action extending generally transversely in the inner surface above the lower open end. A needle bundle is mounted in the housing, which includes one or more needles longitudinally reciprocally movable in the longitudinal channel and are biased towards the inner surface across the grooves to form a capillary gap (such as the gap with gap distance y as illustrated in FIG. 15D) between the needle bundle and the inner surface, for drawing ink out of the ink storage grooves during longitudinal reciprocal movement of the needle bundle across the grooves.

In some embodiments, the ink storage grooves may be closed to the exterior surface of the mouthpiece, and an ink container may be provided and placed in fluid communication with the grooves, such as via an internal fluid conduit though the groove walls. The ink container may be external to the needle module, in which case, the ink container may be connected to the internal fluid conduit by a tube. The ink container may also be installed within the housing in the needle module, or attached to the handle or another part of the ink applicator.

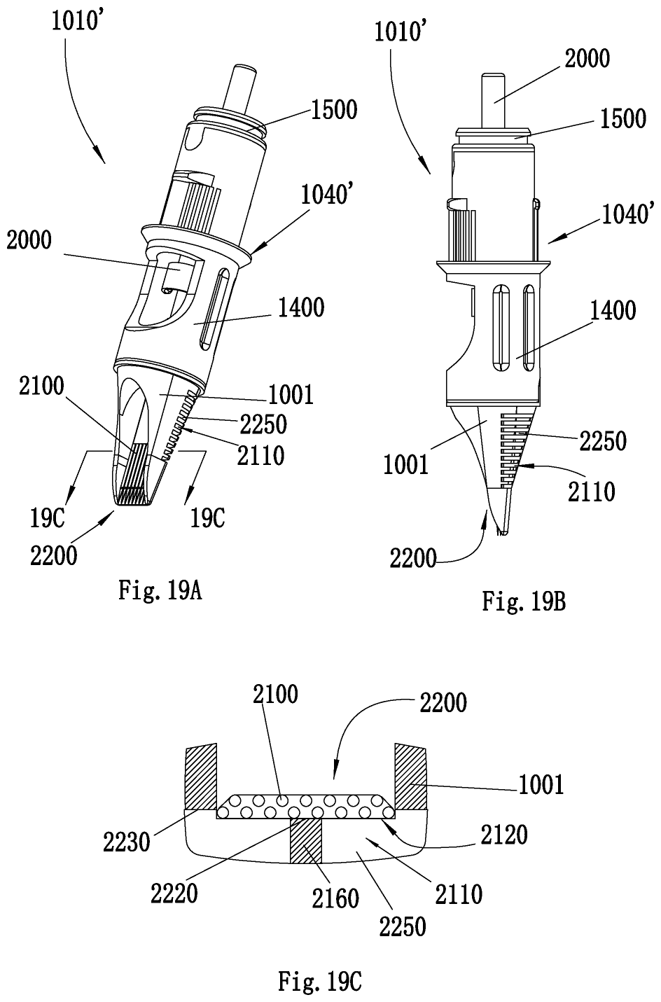

While the above examples are illustrated in the figures with a round tip portion or round needles and needle bundles, in different embodiments, the construction of the needle module may be readily modified or adapted to use flat or Magnum needles. In the latter case, the mouthpiece may typically have a generally rectangular mouth opening, as illustrated in FIGS. 19A, 19B and 19C. The example needle module 1010′ illustrated in these figures has a bundle of 15 needles arranged in two flat rows. The needles may be welded together. The needle module 1010′ includes a needle housing 1040′ for movably mounting a needle bundle 2000 therein. The needle housing 1040′ is formed by a tubular body portion 1400, a mouthpiece 1001 and a cap 1500. Housing 1040′ has a tubular longitudinal channel therein. The channel has an upper open end and a lower open end. The mouthpiece 1001 in needle module 1010′ has ink storage grooves 2110 between groove walls 2250 with contact opening 2120, an opening 2200 with a rectangular profile, and a guide surface 2220. Needle bundle tip portion 2100 is received in opening 2200 and contacts the guide surface 2220, under a biasing force as described earlier with regard to needle module 1010. The ink storage grooves 2110 are separated by a reinforcing wall 2160 and extend from the reinforcing wall 2160 to end surfaces 2230.

As can be appreciated, a needle assembly described herein may be used or adapted to apply other types of liquids to skin. For example, the applied liquid may include colored liquids or pigments, or may include a medicinal or therapeutic agent, collagen, or other like or similar substances. The needle assembly may be used in a liquid applicator for applying the selected liquid.

Other features, modifications, and applications of the embodiments described here may be understood by those skilled in the art in view of the disclosure herein.

### CONCLUDING REMARKS

It will be understood that any range of values herein is intended to specifically include any intermediate value or sub-range within the given range, and all such intermediate values and sub-ranges are individually and specifically disclosed.

It will also be understood that the word “a” or “an” is intended to mean “one or more” or “at least one”, and any singular form is intended to include plurals herein.

It will be further understood that the term “comprise”, including any variation thereof, is intended to be open-ended and means “include, but not limited to,” unless otherwise specifically indicated to the contrary.

When a list of items is given herein with an “or” before the last item, any one of the listed items or any suitable combination of two or more of the listed items may be selected and used.

Of course, the above described embodiments of the present disclosure are intended to be illustrative only and in no way limiting. The described embodiments are susceptible to many modifications of form, arrangement of parts, details and order of operation. The disclosure, rather, is intended to encompass all such modification within its scope, as defined by the claims.

What is claimed is:

1. A tattoo needle tip comprising:
a needle tip body having a lower end and an upper end;
a needle passage defined inside the needle tip body for placing a tattoo needle;
a needle opening defined at the lower end of the needle tip body for a sharp end of the tattoo needle to extend out, the needle opening being communicated with the needle passage;
a capillary ink reservoir receiving and retaining ink therein by capillary action being disposed on the needle tip body and directly connected to the needle opening, the ink reservoir directly feeding ink to the sharp end of the tattoo needle, wherein a plurality of capillary ink containing bodies are provided at the ink reservoir and are directly connected to the needle opening.

2. The tattoo needle tip of claim 1, wherein the ink containing bodies comprise a plurality of capillary grooves extending on an inner surface of the needle opening generally transversal to an axial direction of the tattoo needle.

3. The tattoo needle tip of claim 2, wherein the grooves are distributed along a full longitudinal extent of the needle opening.

4. The tattoo needle tip of claim 1, wherein the ink reservoir comprises a porous material and the ink containing bodies comprise pores in the porous material.

5. The tattoo needle tip of claim 1, wherein the ink reservoir is integrally or detachably formed at the needle tip body.

6. A device comprising a handle and the tattoo needle tip of claim 1 coupled to the handle.

7. A needle assembly comprising the tattoo needle tip of claim 2 and the tattoo needle mounted in the needle passage of the tattoo needle tip.

8. The needle assembly of claim 7, further comprising a biasing member configured to bias the tattoo needle towards the inner surface to reduce a capillary gap between the tattoo needle and the grooves.

9. The needle assembly of claim 8, wherein the biasing member is configured to bias the tattoo needle both longitudinally and transversely towards the inner surface.

10. The needle assembly of claim 8, wherein the biasing member comprises a resilient band, a silicone, latex, or rubber material.

11. The needle assembly of claim 8, wherein the inner surface is shaped to conform to an external profile of the tattoo needle to increase a contact area between the tattoo needle and the ink stored in the grooves.

12. The needle assembly of claim 8, wherein the tattoo needle comprises a needle bundle.

13. The needle assembly of claim 8, wherein the needle tip body comprises a generally cylindrical or conical body having a circumference, and one or more of the grooves extend up to 120° along the circumference of the body.

14. The needle assembly of claim 8, wherein the longitudinal channel has a generally circular profile or a generally polygonal profile.

15. The needle assembly of claim 8, wherein the grooves have a width of about 0.3 mm to about 0.6 mm.

16. The needle assembly of claim 8, wherein the plurality of grooves comprises 3 to 12 grooves.

17. The needle assembly of claim 8, wherein the needle passage comprises a generally conical lower portion and a generally cylindrical upper portion.

18. The needle assembly of claim 8, wherein the grooves are inter-connected.

19. A liquid applicator comprising the needle assembly of claim 8, and a needle actuator for actuating downward movement of the tattoo needle bundle, and a handle coupling the needle actuator to the needle assembly.
